(19) United States
(12) Patent Application Publication
(10) Pub. No.: US 2004/0077573 A1
(43) Pub. Date: Apr. 22, 2004

(54) Method for regulating the activity of an expression product of a gene transferred into living body

(76) Inventors: Hiroki Maruyama, Niigata (JP); Jun-ichi Miyazaki, Osaka (JP); Makoto Sugawa, Shizuoka (JP); Masato Higuchi, Tokyo (JP)

Correspondence Address: FISH & RICHARDSON PC, 225 FRANKLIN ST, BOSTON, MA, 02110, US

(21) Appl. No.: 10/432,305

(22) PCT Filed: Sep. 28, 2001
(86) PCT No.: PCT/JP01/08575

(30) Foreign Application Priority Data
Nov. 24, 2000 (JP) 2000-358389

(51) Int. Cl. A61K 48/00; C12P 21/02; C07K 14/52; C12N 15/87

(52) U.S. Cl. 514/44; 435/459; 435/69.1; 435/320.1; 435/325; 435/69.5; 530/351

(57) ABSTRACT

The activity of an expression product of a gene introduced into a living body can be regulated by the coexistence of a protein (interfering substance) that interferes with the activity of the expression product. Receptors of the expression product and such may be used as the interfering substance. The activity of an overexpressed product of a gene introduced in gene therapy can be regulated. The administration of an interfering substance can be achieved by in vivo expression of the gene encoding the substance.

### TECHNICAL FIELD

The present invention relates to a method for regulating the activity of an expression product of a gene artificially introduced into a living body.

### BACKGROUND ART

Human gene therapy was tried for the first time in 1990 in the US, and thereafter has been widely conducted in many countries. It is expected that, once the background of diseases are understood at the gene level, gene therapy can be used for treating many diseases by regulating the expression of the genes causing the diseases. The most-widely conducted gene therapy at present is a type of therapy wherein the activity of a protein, which is deficient due to abnormalities in a gene, is compensated by introducing a normal gene. An example of this type of gene therapy is a therapeutic method for adenosine deaminase (ADA) deficiency, in which an ADA expression vector is introduced. Similar gene therapies include the introduction of normal p53 into cancer cells. This gene therapy attempts to treat cells in which cancerization cannot be suppressed due to mutations in the p53 gene by the action of normal p53.

In early gene therapies, the expression level of the artificially introduced gene was often lower than expected. Therefore, investigations into -means to accomplish stronger expression of the introduced gene by improving vector and methods for introducing the gene have attracted attention as a new subject. However, with the progress of these studies, some are worried about a long-term increase in the expression of the introduced gene at significantly higher levels than the physiological level. The expression mechanism of an artificially introduced gene is not accompanied by a feedback mechanism that suppresses overexpression. Thus, a strong expression system always bears the problem of overexpression. Maintained expression of an artificially introduced gene may cause side effects during therapy.

For example, a type of hematopoietic hormone, erythropoietin (abbreviated hereinafter as EPO), is effective for the treatment of anemia. However, excessive EPO may cause plethora. Gene therapy using vascular endothelium growth factor (VEGF) is carried out to treat pain during walking (intermittent claudication), arteriosclerosis obliterans showing histological damage in the lower limb, ischemic cardiac diseases including chronic cardiac failure, restenosis, and such. In gene therapy of angiogenesis by VEGF, excessive VEGF may cause angiogenesis in many other sites in addition to the target site. Upon acceleration of angiogenesis, the possibility of abnormal development of tissues, risks of increase in malignant tumors in tumor bearing patients, and such may arise. Therefore, the expression level of an introduced gene and the activity of an expression product need to be controllable for future gene therapy.

An example of a method for regulating the expression level of an introduced gene includes the method wherein a regulatory region reacting with a drug is integrated into a vector to be introduced and then administering the drug (Ye et al., Science, 1999, 283, 88-91). According to this method, however, problems arise, such as side effects caused by the administered drug. Therefore, there is a need for safer and more reliable methods for regulating the expressed gene.

Meanwhile, various cytokines have recently been revealed to be involved in-diseases, and clinical application of anticytokine therapy, such as administration of soluble chimeric receptors or antibodies against these cytokines, is in progress and already has accomplished results (Feldman AM et al., The role of tumor necrosis factor in the pathophysiology of heart failure, J. American College of Cardiology, 2000, 35, 537-544; Boehme M W & Gao I K, Present importance of direct immunologically based intervention strategies using anticytokines in rheumatoid arthritis, Zeitschrift fur Rheumatologie, 1999, 58, 251-166; Choy E H et al., Monoclonal antibody therapy in rheumatoid arthritis, British J. Rheumatology, 1998, 37, 484-490).

For example, chimeric receptors of tumor necrosis factor-α (TNF-α) or antibodies against TNF-α have been administered against TNF which is indicated to be involved in Crohn's disease, heart diseases (Feldman A M et al., The role of tumor necrosis factor in the pathophysiology of heart failure, J. American College of Cardiology, 2000, 35, 537-544), and rheumatism (Ohshima S et al., Long-term follow-up of the changes in circulating cytokines, soluble cytokine receptors, and white blood cell subset counts in patients with rheumatoid arthritis (RA) after monoclonal anti-TNF α antibody therapy, J. Clin. Immunology, 1999, 19, 305-313; Choy E H et al., Monoclonal antibody therapy in rheumatoid arthritis, British J. Rheumatolbgy, 1998, 37, 484-490), and have been achieving clinically effective results. Furthermore, an inhibitory effect on hepatic fibrosis in an animal model was obtained by the administration of chimeric IgG-soluble receptor comprising an extracellular portion of TGF-β type II receptor indicated to be involved in fibrosis (George J et al., In vivo inhibition of rat stellate cell activationby soluble transforming growth factor β type II receptor: a potential new therapy for hepatic fibrosis, Proc. Natl. Acad. Sci. USA, 1999, 96, 12719-12724). In these reports, components which interfere with the activity of cytokines are administered in order to regulate the activity of the cytokines that are the cause of diseases.

In gene therapy wherein an adenovirus vector is administered, anti-CD4 monoclonal antibodies have also been administered to animals to suppress the immune response against the virus. As a result, effects such as sustained reporter expression of the introduced gene, decrease in the antibody titer against the virus, and suppression of IFN-γ increase have been achieved (Schroeder G et al., Immune response after adenoviral gene transfer in syngeneic heart transplants: effects of anti-CD-4 monoclonal antibody therapy, Transplantation, 2000, 70, 191-198). These results suggest the effectiveness of the anti-CD4 antibody therapy in organ transplantation, etc.

Furthermore, the administration of an antibody against CD45, involved in the activation of lymphocytes; has been shown to inhibit the onset of experimental allergic encephalitis (EAE), suppress the proliferation of T cells, and significantly suppress the production of not only TNF-α but also Th-1 cytokines, such as IFN-γ and IL-2 (Schiffenbauer J et al., Prevention of experimental allergic encephalomyelitis by an antibody to CD45RB, Cellular Immunology, 1998, 190, 173-182). However, these methods, wherein an antibody or a recombinant type protein of a soluble receptor against a target cytokine is administered, are considered to have problems, such as reduction in the effect of repeated administration due to antibody production against administered antibody or recombinant protein and costs of the recombinant protein.

### DISCLOSURE OF THE INVENTION

The objective of the present invention is to provide a method for regulating the activity of an expression product of a gene artificially introduced into a living body.

The feedback mechanism inherent in a living body does not function on a gene artificially introduced into the living body. Accordingly, the present inventors conceived that a certain kind of feedback mechanism could be realized by administering a compound capable of regulating the activity of an artificially introduced protein in vivo.

Finally, the present inventors revealed that the activity of an expression product of a gene artificially introduced into a living body can be regulated by allowing a substance that interferes with the activity of the expression product to coexist with the gene in vivo, and completed the present invention. Specifically, the present invention relates to a method for regulating the activity of an expression product of a gene introduced into a living body, as well as a vector used therefor, as follows:

[1] a method for regulating the activity of an expression product of a gene introduced into a living body involving the coexistence of a protein that interferes with the activity of the expression product;

[2] the method according to [1], wherein the coexistence of the protein that interferes with the activity of the expression product is achieved by expressing a gene encoding the protein in the same living body;

[3] the method according to [2], wherein the gene encoding the protein that interferes with the activity of the expression product is introduced into the living body via an expression vector consisting of a naked DNA;

[4] the method according to [3], wherein the expression vector is introduced into the living body by electroporation;

[5] the method according to [1], wherein the protein that interferes with the activity of the expression product is a protein selected from the group consisting of: a receptor of the expression product, an activity-neutralizing antibody of the expression product, and a protein containing an active site of the receptor or the antibody;

[6] the method according to [2], wherein the gene encoding the protein that interferes with the activity of the expression product is expressed under the control of an inducible promoter;

[7] a method for regulating the activity of an expression product of a gene introduced in gene therapy by the method according to [1];

[8] the method according to [7], wherein the introduced gene encodes a protein which belongs to a group selected from blood proteins, cytokines, and hormones;

[9] the method according to [8], wherein the protein belonging to a group selected from blood proteins, cytokines, and hormones is selected from the group consisting of:

erythropoietin, granulocyte-colony stimulating factor, granulocyte-macrophage colony stimulating factor, stem cell factor, thrombopoietin, tumor necrosis factor-ax, transforming growth factor-β, CD4, CD45, bone morphogenetic protein, interferon-α, interferon-γ, interleukin-1, interleukin-2, interleukin-6, interleukin-11, interleukin-12, epidermal growth factor, acidic fibroblast growth factor, basic fibroblast growth factor, fibroblast growth factor-I, platelet derived growth factor, vascular endothelial growth factor, nerve growth factor, brain derived neurotrophic factor, insulin-like growth factor-I, insulin-like growth factor-II, human growth hormone, parathyroid hormone, angiostatin, pigment epithelial cell derived factor, and hepatocyte growth factor;

[10] the method according to [9], wherein the protein is erythropoietin;

[11] the method according to [10], wherein the protein that interferes with the activity of the expression product is a solubilized erythropoietin receptor;

[12] the method according to [1], wherein the protein that interferes with the-activity of the expression product is a fusion protein with another protein;

[13] an expression vector for regulating the activity of an expression product of a gene introduced into a living body, which can express a protein that interferes with the activity of the expression product in the same living body;

[14] the expression vector according to [13], wherein the protein that interferes with the activity of the expression product is a fusion protein with another protein;

[15] a composition for regulating the activity of an expression product of a gene introduced into a living body, which comprises the expression vector of [13] as an active ingredient;

[16] use of an expression vector that can express a protein which interferes with the activity of an expression product of a gene introduced into a living body in the same living body, in the production of a composition for regulating the activity of the expression product of a gene introduced into a living body; and

[17] the use of the expression vector according to [16], wherein the protein that interferes with the activity of the expression product is a fusion protein with another protein.

Herein, the phrase “gene introduced into a living body” refers to any gene which is artificially introduced into a living body for any purpose. For example, a representative gene includes those that are artificially introduced aiming gene therapy. In addition to therapeutic purposes, the introduction of a foreign gene is often attempted to introduce traits not inherent in a subject organism. For example, a gene introduced for the purpose of producing a useful protein in an animal is also included as the gene artificially introduced into a living body in the present invention.

This invention is particularly useful for genes whose purpose of introduction is achieved by expressing the gene product thereof in the blood of animal. As compared with locally limited overexpression of a product, an expression product existing in blood exerts systemic influence. Therefore, regulation of the activity of a protein encoded by a gene that is artificially introduced into a living body becomes particularly important when the gene expression product is produced into the blood of an animal. In the present invention, genes that are introduced to supply their expression products into blood include genes encoding proteins belonging to blood proteins, cytokines, or hormones. The term “blood protein” refers to various protein contained in blood, such as albumin, globulin, ferritin, and various enzymes. Examples of cytokines and hormones specifically include the following proteins:

erythropoietin (EPO);

granulocyte-colony stimulating factor (G-CSF);

granulocyte-macrophage colony stimulating factor (GM-CSF);

stem cell factor (SCF);

thrombopoietin (TPO);

tumor necrosis factor-a (TNF-α);

transforming growth factor-α (TGF-β);

CD4;

CD45;

bone morphogenetic protein (BMP);

interferon-α (IFN-α);

interferon-γ (IFN-γ);

interleukin-1 (IL-1);

interleukin-2 (IL-2);

interleukin-6 (IL-6);

interleukin-11 (IL-11);

interleukin-12 (IL-12);

epidermal growth factor (EGF);

acidic fibroblast growth factor (aFGF);

basic fibroblast growth factor (bFGF);

fibroblast growth factor-I (FGF-I);

platelet derived growth factor (PDGF);

vascular endothelial growth factor (VEGF);

nerve growth factor (NGF);

brain derived neurotrophic factor (BDNF);

insulin-like growth factor-I (IGF-I);

insulin-like growth factor-II (IGF-II);

human growth hormone (hGH);

parathyroid hormone (PTH);

angiostatin;

pigment epithelial cell derived factor (PEDF); and

hepatocyte growth factor (HGF).

Herein, the phrase “protein that interferes with the activity of a gene expression product” refers to proteins that can modify the activity of the gene expression product through a certain mechanism. The protein that interferes with the activity, herein, is referred to as the “interfering substance”. Furthermore, “regulation” as used in the instant specification means to modify the activity of the gene expression product to a desired level by altering the amount of the interfering substance. The term “regulation” herein includes not only down-regulation but also up-regulation of the activity. Specifically, interfering substances include proteins that interfere with the activity through. binding, decomposition, competition, and such. More specifically, the interfering substances include receptors of the gene expression product, antibodies capable of inhibiting the binding between the gene expression product and its receptor by blocking a receptor-binding moiety of the gene expression product, and proteins acting as an antagonist of the gene expression product.

For example, for EPO, inferring substances of the present invention include EPO receptor, antibodies capable of inhibiting the binding between EPO and its receptor by blocking a receptor-binding moiety of EPO, and proteins acting as an antagonist of EPO. Similarly for other cytokines, such as VEGF, receptors whose ligand is the cytokine, or antibodies thereto can be used as the interfering substance.

These proteins can be active fragments thereof or fusion substances of an-active fragment with other molecules, so long as they can interfere with the activity of the gene expression product. For example, a ligand-binding region of a receptor or a fusion protein containing the binding region can be used as an interfering substance. Furthermore, a fragment of an antibody containing its antigen-binding region, or a single-strand antibody can be also used. A fusion substance includes a fusion of the receptor and an antibody Fc region. By fusing the protein with the antibody Fc region as the interfering substance, effects such as follows can be expected:

1) elongated retention time in blood through the suppressed degradation of the interfering substance due to its large molecular weight; and

2) enhanced affinity for target molecules due to dimerization of the interfering substance.

When the receptor is a heterologous protein to the host, in vivo stability of the receptor is expected to be enhanced by its chimerization with a host protein. This fusion substance can be introduced into the living body by expressing a chimeric gene encoding the fusion substance.

An interfering substance is preferably derived from an organism which is the subject of administration. Therefore, when the method of the present invention is applied to humans, a protein derived from human is preferably used. Using a protein derived from the same organism, a safer formulation that avoids induction of the attack by the immune mechanism can be obtained. Furthermore, such interfering substance would not be eliminated through the immune mechanism, and thus effective administration can be expected.

When an antibody or a fragment thereof is used as an interfering substance, its safety can be enhanced by substituting a part thereof with a structure of human immunoglobulin. Specifically, a part of immunoglobulins derived from non-human species can be humanized by techniques such as chimeric antibody production and complementarity determining region (CDR) grafting. A humanized protein obtained in this manner is preferable as the interfering substance when the present invention is applied to humans.

In accordance with the observed overexpression of the gene product, an interfering substance of the present invention is administered into a site where the gene product is present. For example, when overexpression of the gene product is observed in blood, the interfering substance is administered orally or parenterally so that it can be present in blood in an effective amount to regulate the activity of the initial gene product. The administration method is not particularly limited. Increased retention time in blood of the interfering substance of the present invention can be expected by administering it as a binding product with a polymerized compound. The polymerized compound may include hyaluronic acid, polyethylene glycol, albumin, and such.

In the present invention, gene therapy can be used to administer an interfering substance consisting of a protein. Specifically, DNA encoding the interfering substance can be integrated into a known vector for administration into living body. A protein, the interfering substance, derived from the same specie as that to which the gene is administered express the interfering substance in the living body is safe, because it does not stimulate the immune mechanism. Furthermore, an accurate and continuous effect of the interfering substance can be obtained due to its sustained production in the living body without being attacked by the immune mechanism. Moreover, a safe preparation for gene therapy can be produced inexpensively with smaller production facilities than industrially producing and purifying proteins.

For in vivo coexistence of an interfering substance by administration and expression of a DNA encoding it, the interfering substance is preferably expressed so that it is provided to a site where a protein that should be regulated by the interfering substance is present. For example, when regulating the activity of a protein in blood, the interfering substance can be expressed as a secretory protein. Herein; the interfering substance expressed as a secretory protein is referred to as “soluble protein”.

When a membrane protein, such as a receptor, is used as an interfering substance, its ligand-binding region can be expressed as a secretory protein. More specifically, when an EPO receptor. is used for EPO, DNA encoding an amino acid sequence comprising the N-terminal amino acid residues at position 1 to 222 but not the transmembrane region of the EPO receptor can be used. A secretion signal is further added to the N-terminus of a protein consisting of such amino acid sequence to express it as a secretory protein. For example, secretion signal and ligand-binding region of the human EPO receptor is included within position 1 to 246 of the precursor protein encoded by cDNA of the human EPO receptor. The secretion signal added to the N-terminus can be an amino acid sequence derived from either the EPO receptor or other species that can function in vivo. Herein, the EPO receptor expressed as a secretory protein as described above is referred to as “soluble EPO receptor”. A DNA encoding an interfering substance can be introduced into a living body by direct administration of a virus vector or DNA. Many virus vectors, such as those using retrovirus, lentivirus, adenovirus, and adeno-associated virus (AAV) have been developed. “The direct administration of DNA” is a method wherein a viral structure-free expression vector for eucaryotic cells is administered into a living body in vivo, in situ, or ex vivo. Many methods have been performed for artificially introducing foreign genes into a living body. The present invention can be applied to expression products of any gene artificially introduced by known gene introduction methods.

Specifically, vectors -shown below may be used for, the purpose of introducing a gene into a living body. Any of these vectors can be used for the introduction of DNA encoding an interfering substance of the present invention.

Plasmid expression vectors: pVR, pCMV, and pCAGGS

Duplication defective adenoviruses: AdEF1 and ADMLP

Retrovirus vector: LrEPSN Adeno

Adeno associated virus vectors: rAAV-ET and AdCMV

Among these various vectors, virus vectors such as retrovirus, adenovirus, and adeno-associated virus may cause a phenomenon wherein other cells are infected with the introduced vector. This phenomenon, called the “bystander effect”, results in sustained expression of an introduced gene and thus is effective for obtaining strong expression. However, the introduction of an interfering substance in the present invention is intended to regulate the activity of an expression product of a gene introduced for therapy. Therefore, sustained expression over the expected level may act competitively with the therapeutic effect.

However, by adopting an inducible promoter for regulating the expression of an interfering substance, a virus vector can be utilized in the present invention. Specifically, the expression of the interfering substance can be regulated by administering a specific drug utilizing a promoter whose expression is induced by the drug. The use of such a virus vector, even if continuous infection of the vector occurs, can prevent unnecessarily high suppression of the activity of a gene expression product to be regulated by the interfering substance.

Thus, gene therapy using an expression vector, consisting exclusively of minimum elements including an expression regulatory region and a gene to be introduced, is a preferable method for introducing DNA encoding an interfering substance of the present invention.

A general expression vector is composed of expression regulatory regions, such as a replication origin, promoter, enhancer, and polyadenylating sequence; a foreign gene to be expressed; and so on. A method using such expression vectors in place of a virus vector is called the “naked DNA method”. Herein, a “naked DNA” is defined as an expression vector consisting exclusively of DNA.

Therefore, a naked DNA is free of biological structures, such as proteins and sugars, that are observed in virus vectors. According to the naked DNA method, a gene is transiently expressed, no spontaneous intercellular infection of a vector (bystander effect) occurs, and the method implies no risk that the vector may be integrated into the host chromosome; which makes the method preferable for the present invention. Furthermore, the naked DNA is advantageous in that a larger amount of the vector can be prepared at lower costs as compared with viral vectors. Any of these methods for introducing a gene can be used as the method for introducing DNA encoding an interfering substance of the present invention.

To utilize the naked DNA method for expressing an interfering substance of the present invention, for example, an expression vector as follows is constructed. Specifically, the expression vector is composed of a DNA encoding the interfering substance, a promoter region, a 3′-region defining a transcription termination signal, a polyadenylating region, and so on. The promoter region should function in the cell or living body targeted to express the DNA encoding the interfering substance.

The promoter region may be derived from, for example, other organisms or other genes so long as it allows expression of the DNA encoding an interfering substance. For example, promoters derived from other eucaryotic genes-or viral genes may be used. So long as the promoter can express the DNA encoding the interfering substance, the promoter does not have to be specific. When an inducible promoter is used, the expression thereof can be regulated by the induction condition.

Alternatively, when a promoter having tissue specificity is used, an interfering substance can be expressed specifically in a certain organ. Accordingly, such promoter enables application of the method for expressing the interfering substance in a tissue which is actually damaged by the over expression of a gene expression product. For example, when the oxygen supply in a tissue is saturated due to overexpression-of EPO, an artificial feedback loop can be constructed by integrating a promoter region responding to high oxygen concentration upstream of the introduced gene encoding the interfering substance. Specifically, promoters of following genes can be used.

Non-specific promoters:

Chicken β-actin, smooth muscleα-actin, thymidine kinase,

metallothionein, interferon, and immunoglobulin

Inducible promoters: steroid

steroid hormone receptors

Virus-derived promoters:

SV40, hepatitis B virus, and adenovirus major late gene

The promoter may be modified by introduction of a nucleotide sequence constituting another expression regulatory region. For example, the gene transcription efficiency of the chicken β-actin promoter is known to be improved by modifying a part thereof with rabbit β-globulin gene-derived splicing acceptor (Japanese Patent No. 2824434 (1998)).

The expression vector may contain a terminator and an enhancer, in addition to a promoter. These expression regulatory regions can be derived not only from the gene to be introduced, but also from other genes or other species, as is the case with the promoter.

A naked DNA capable of expressing DNA encoding an interfering substance can be introduced into a living body by known methods. For example, ex vivo introduction of a DNA is performed by. transplanting skin cells or blood cells introduced with an expression vector into a living body. When skin cells are transplanted, the expression level of the interfering substance can be easily regulated by removing the skin cells. The introduction of the expression vector into cells can be conducted by the electroporation method or such. The introduction of the naked DNA into the living body by electroporation is carried out, for example, in the following manner. Specifically, after intramuscular injection of the DNA into the limb, electrodes are set so that the injected site is sandwiched between them, and voltage is applied by pulsing according to the electroporation method. To introduce a naked DNA into an organ transplant, the DNA can be efficiently introduced with less damage to tissues by the electroporation method wherein the organ injected with the DNA is soaked in a solution, such as physiological saline, and the whole solution is stimulated with pulses without contacting the organ directly with electrodes. Alternatively, a DNA encoding the interfering substance can be expressed in organs, such as liver and kidney, by intravenous administration according to the hydrogel method. The gene introduction by intravenous administration using TransIT In Vivo Gene Delivery System (Mirus Corporation) or such can also be used.

Moreover, for introducing a DNA encoding an interfering substance, the hemagglutinating virus of Japan (HVJ)-liposome method having both characteristics of easily-handled liposome method and highly-efficient virus vector method may be used. According to this method a DNA can be efficiently introduced and expressed in even nondividing cells, and the DNA is expressed transiently without being introduced into the host genome. Accordingly, it can be said that, similar to the naked DNA, this method is a preferable expression system for the DNA encoding the interfering substance of the present invention.

An introduced DNA as described above encoding an interfering substance is expressed in introduced cells to produce the interfering substance in vivo. The produced interfering substance suppresses the activity of an expression product of an artificially introduced gene. The expression of the interfering substance is reduced and disappears in accordance with the life span of the transformed cells, thus does not unnecessarily reduce the activity of the expression product of the artificially introduced gene.

### BRIEF DESCRIPTION OF THE DRAWINGS

FIG. 1 is a schematic illustration showing the structure of expression vectors of the present invention.

FIG. 2 is a graph showing the changes in the hematocrit value in blood of rats introduced with pCAGGS-EPO and pCAGGS. The hematocrit value (%) is shown on the vertical axis, and the time course (weeks) after the introduction of pCAGGS-EPO is shown on the horizontal axis. In the graph, *: p<0.05, **: p<0.01, ***: p<0.001, and ****: p<0.0001.

FIG. 3 is a graph showing the changes in the hematocrit level in blood. The hematocrit value (%) is shown on the vertical axis, and the time course (weeks) on the horizontal axis. In each group, n=9. It the graph, *: p<0.05, ***: p<0.001., ****: p<0.0001, #: p<0.05, ##: p<0.01, ###: p<0.0001, ####: p<0.0001, &: p<0.05, &&: p<0.001.

FIG. 4 is a graph showing the changes in the number of reticulocytes in blood. The number of reticulocytes (×10⁴/μl) is shown on the vertical axis, and the time course (weeks) on the horizontal axis. In each group, n=9. In the graph, *: p<0.05, **: p<0.01, ***: p<0.001,. ****: p<0.0001, #: p<0.05, ###: p<0.0001, ####: p<0.0001, &&&: p<0.001.

### BEST MODE FOR CARRYING OUT THE INVENTION

This invention is specifically illustrated below with reference to Examples, but it is not to be construed as being limited thereto.

The animals used in the Examples were 8-weeks-old male rats (SD, Charles River Inc., Tokyo) kept for one week or more in a light-dark (each 12 hours) cycle under pathogen-free conditions before starting the experiment. All the animals were supplied with water and standard food (MF containing 23.8% protein; 0.24% sodium; 0.0154% iron, Oriental Yeast Co., Ltd., Tokyo) ad libitum.

### EXAMPLE 1

### Preparation of Plasmid Vectors:

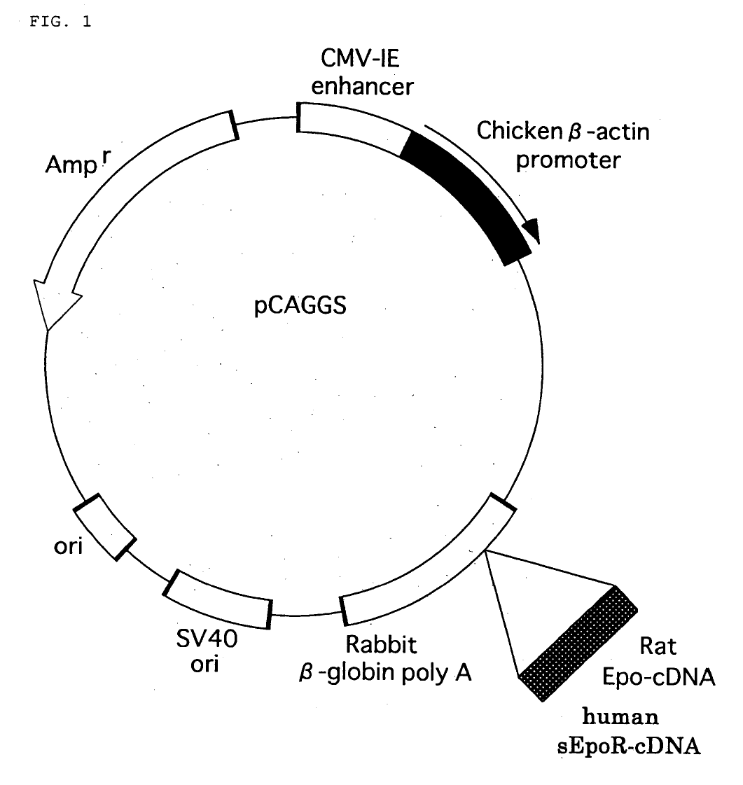

Plasmid pCAGGS-Epo was constructed by inserting rat EPO cDNA into the XhoI site of expression vector pCAGGS (Niwa H et al., Gene, 1991, 108, 193-199). Plasmid pCAGGS-hSEPOR2 was constructed by inserting a cDNA of human soluble EPO receptor (hSEPOR2, WO 99/53313) into the XhoI site of the expression vector pCAGGS in the same manner as described above. Each plasmid was purified by Qiagen EndoFree plasmid Giga kit (Qiagen GmbH, Hilden, Germany) (Maruyama H et al., Human Gene Therapy, 2000, 11, 429-437). The structure of the expression vectors constructed in this Example is shown in FIG. 1. The nucleotide sequences encoding the human soluble EPO receptor and the rat EPO are described in SEQ ID NOs: 1 and 2, respectively.

As the control, plasmid pCAGGS was used. The resulting DNA was dissolved in sterilized phosphate-buffered saline (PBS) and prepared to a concentration of 2 μg/μl for the injection.

### EXAMPLE 2

### Introduction of pCAGGS-Epo and pCAGGS:

Intramuscular injection of the plasmid DNA by the electroporation method was conducted according to previously-reported method (Maruyama H et al., Human Gene Therapy, 2000, 11, 429-437). Specifically, fifty μg of plasmid DNAs, pCAGGS-Epo and pCAGGS, were injected into both the medial and lateral sites of the right and left lower limbs (200 μg in total), respectively. Then, a pair of stainless steel electrodes of 10 mm width and 5 mm length were set so that the DNA-injected site is located in the center of the two electrodes, and electric pulse generator (Electro Sequre Porator T820; BTX) was used to give electric pulses (8 pulses, duration 50 msec, 100 V, 1 pulse/s).

After the introduction of the gene, the animals were lightly anesthetized with diethyl ether, 2 ml blood was collected from the heart according to the method of Ohwada et al. (Ohwada K., Exp. Anim., 1986, 35, 353-355), and the EPO level in serum was measured using Recombigen EPO kit (Nippon DPC, Chiba). The erythrocyte, hematocrit, hemoglobin, leukocyte, and platelet were measured with Sysmex SE-900 (Sysmex, Hyogo) and the reticulocyte with Sysmex R-3500.

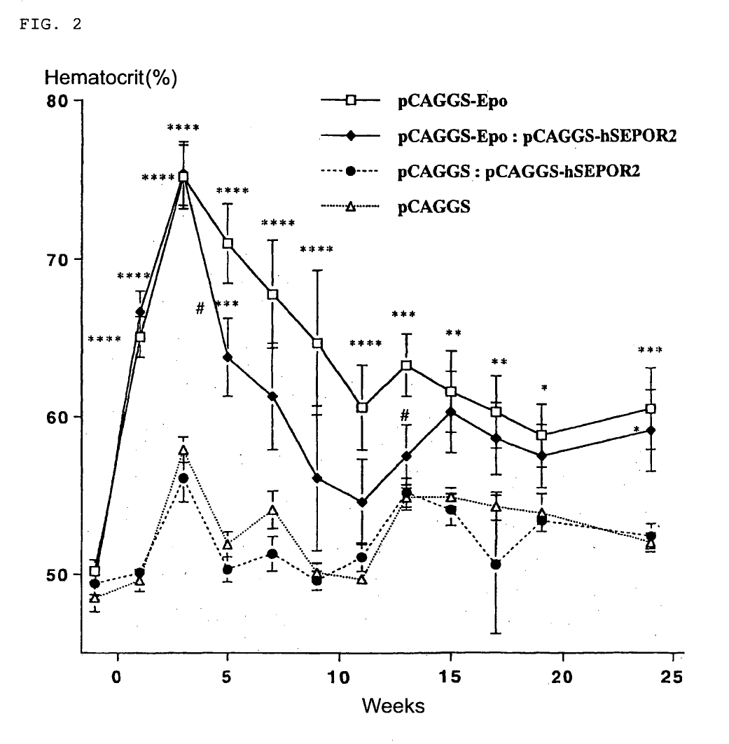

The results are shown in FIG. 2. The experimental results are shown by mean ± standard error, and the statistically significant difference was evaluated by a t-test without correspondence, and p<0.05 was regarded as significant. Upon the introduction of the EPO gene, the hematocrit value increased and reached almost the peak after 3 weeks (FIG. 2). On the other hand, the group introduced with the control plasmid DNA showed an increase in the hematocrit value 4 weeks after the introduction, but the amount of increase was very slight as compared with that of the group introduced with the EPO gene.

### EXAMPLE 3

### Introduction of pCAGGS-hSEPOR2

The animals confirmed to have an increased hematocrit value in Example 2 were transformed with the human EPO soluble receptor expression vector (pCAGGS-hSEPOR2) on the fourth week. pCAGGS-hSEPOR2 was intramuscularly injected in the right and left tights in the same manner as in Example 2 at an amount of 800 μg in total. Electroporation and blood collection were also conducted in the same manner as in Example 2.

The hematocrit value of the group introduced with the human EPO soluble receptor expression vector tended to decrease compared with that of the group without introduction, and a significant reduction in the hematocrit value was confirmed on the fifth and thirteenth weeks. When the EPO soluble receptor was introduced to the control group after 4 weeks, the hematocrit value did not change thereafter to be further reduced, and caused no-anemic conditions.

According to the results described above, excessive action of a gene introduced for therapeutic purpose can be reliably regulated by introducing a soluble receptor into the living body. Furthermore, at the time when the expression level of the introduced gene was reduced (on the 15 weeks or later in this experiment) or under-normal conditions such as that shown in the control wherein the expression level-of the foreign gene was zero, no strong side effects that may further lower the expression level was confirmed. Thus, the present method was considered to be a highly safe regulation method.

### EXAMPLE 4

### Regulation of Rat EPO Levels by Introducing Human EPO Receptor-IgG Fc Chimeric Gene

In the above Examples, the action of rat EPO was suppressed by the human soluble EPO receptor. In this Example, changes in the action of rat EPO were detected using, in place of the human soluble receptor, a chimeric protein thereof with rat IgGFc.

IgG1Fc cDNA was cloned from commercially-available rat spleen library (Quick Clone, Clontech) by the PCR method. The IgG1Fc cDNA was inserted into the expression vector pCAGGS in the same manner as in Example 1 to construct rat IgGFc expression plasmid pCAGGS-IgG1Fc. In addition, a chimeric cDNA, IgG1Fc cDNA ligated with hEPOR, was inserted into the XhoI site of the expression vector pCAGGS in the same manner as in Example 1 to construct chimeric protein expression plasmid pCAGGS-hSEPOR2-IgG1Fc. The nucleotide sequence of the insert in the chimeric protein expression plasmid pCAGGS-hSEPOR2-IgG1Fc is shown in SEQ ID NO: 3. Each plasmid was purified as in Example 1 and used in the following experiment.

100 μg pCAGGS-Epo was intramuscularly injected by the electroporation method in the medial and lateral sites of the right and left lower limbs of an animal (rat) (400 μg in total). pCAGGS-Epo is a plasmid expressing rat Epo. One and two-weeks after the gene introduction, increase in EPO levels in blood were confirmed, respectively, and3 weeks later, 800 μg of each plasmid, pCAGGS-IgG1Fc, pCAGGS-hSEPOR2, and pCAGGS-hSEPOR2-IgG1Fc, were introduced via a tail vein into the rat using TransIT® In Vivo Gene Delivery System (MIR5125, Mirus) (Liu F, Song Y K, Liu D, Hydrodynamics-based transfection in animals by systemic administration of plasmid DNA, Gene Therapy, 1999, 6, 1258-1266).

Blood was collected in the course of time, and the EPO level, the hematocrit value, and the number of reticulocytes in blood were measured for the following 24 weeks. For statistically significant difference test, t-test without correspondence-was carried out, and level of significance of 5% or less was regarded as significant.

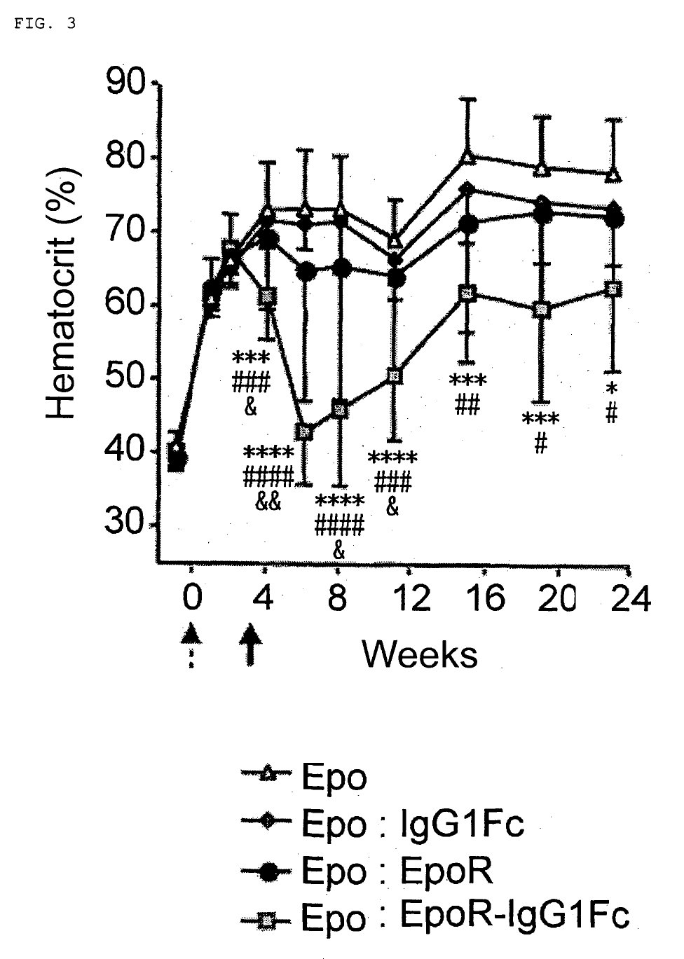

Animals with increased EPO levels upon the introduction of the plasmid pCAGGS-hEPO were grouped such that each group had a similar degree of increase, and each of the soluble human EPO receptor was introduced via a tail vein. As a result, the serum EPO level reached the peak after 1 week due to the introduction of EPO, and then gradually decreased (data not shown). The hematocrit value increased immediately after the introduction, reached a peak after 4 weeks, and then remained almost constant. Thereafter, though not significant to the value of the initial peak, the level increased again after 16 weeks and this increased level was maintained even after 24 weeks (FIG. 3).

On the other hand, the group introduced with the hSEPOR2-IgG1Fc chimeric protein expression plasmid showed a significant decrease in the hematocrit value from the fourth week to the-twenty-fourth week, as compared with that of the group introduced with EPO (FIG. 3). The group into which IgG1Fc or soluble human EPO receptor had been introduced showed a tendency to lower the hematocrit value as compared with the group introduced with the EPO alone, but their difference was not significant. The action of the hSEPOR2-IgG1Fc chimeric protein was also significant compared with the IgG1Fc introduced group, and also showed a significantly stronger effect from the fourth week to the twelfth weeks in comparison with the soluble human EPO receptor introduced group (FIG. 3).

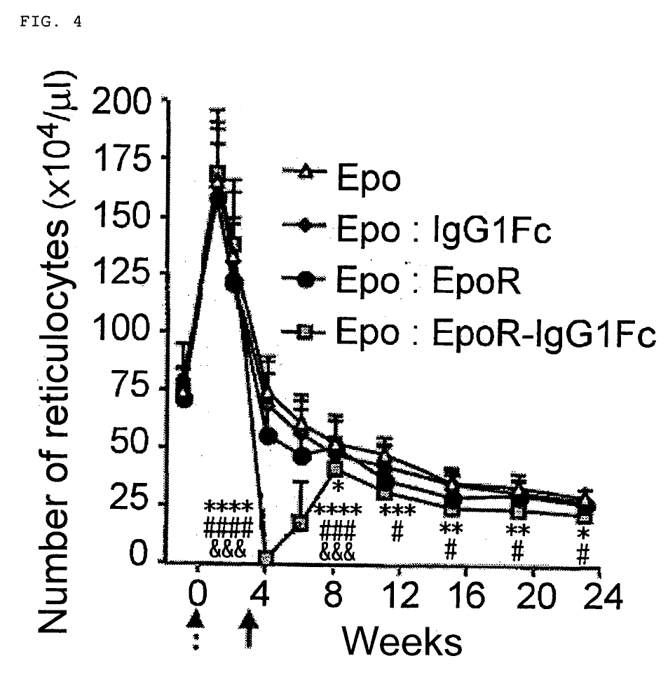

The number of reticulocytes reached the peak one week after the introduction of EPO and rapidly decreased thereafter. No change was observed in the reticulocyte level by the introduction of IgG1Fc or EPO receptor as compared with the group introduced with the EPO gene alone. However, a significant decrease in the number of reticulocytes from the fourth week to the twenty-fourth week was observed by the introduction of the hSEPOR2-IgG1Fc chimeric protein. This action was significantly stronger than that in the groups introduced with IgF1Fc and EPO receptor, respectively (FIG. 4).

As a result, increased EPO level in blood is more strongly reduced by introducing the chimeric receptor with rat IgG1Fc (hSEPOR2-IgG1Fc) than by expressing the EPO receptor alone.

The introduction of the EPO receptor in Example 2 (FIG. 2) significantly reduced the increased hematocrit value. However, in Example 4 (FIG. 3), despite the same tendency was observed the difference was not significant. This difference in the effect of the EPO receptor was ascribed to the difference in the introduced amount of rat Epo (pCAGGS-EPO). The introduced pCAGGS-EPO amount was 200 μg in Example 2 (FIG. 2), while it was 400 μg in Example 4 (FIG. 3). That is, the same amount of EPO receptor as in Example 2 may have failed to sufficiently neutralize the activity of EPO in Example 4, due to the fact that the EPO is expressed at a larger amount in Example 4 than in Example 2.

### Industrial Applicability

This invention realizes a method for regulating the activity of an expression product of an introduced gene in vivo, which was unconsidered in conventional gene therapy. Constant expression of, a gene artificially introduced into a living body at a higher level than the physiological level can cause side effects in vivo. For example, when the introduced gene encodes EPO, it causes side effects such as plethora.

According to the present invention, side effects caused by overexpression can be reduced by administering an interfering substance. The Examples exemplify the administration of the interfering substance via the introduction of a gene encoding a soluble receptor. Apart from receptors, similar effects can be achieved by introducing a neutralizing antibody or a gene encoding the antibody.

The method of administering a recombinant soluble receptor protein or an antibody protein, however, requires a large amount of the purified proteins and repetitive administration to achieve an inhibitory action, which increases cost and physical burden, such as regular treatment in the hospital, on patients. The method of introducing a gene demonstrated by the present inventors is excellent in terms of the durability of the effect and safety, since the protein is produced in cells of tissues of the patient himself and thus has less antigenicity with respect to sugar chain modification and such. Furthermore, when the gene is introduced into the skin, the introduced gene can be easily switched on/off by transplanting the skin.

1. A method for regulating the activity of an expression product of a gene introduced into a living body involving the coexistence of a protein that interferes with the activity of the expression product.

2. The method according to claim 1, wherein the coexistence of the protein that interferes with the activity of the expression product is achieved by expressing a gene encoding the protein in the same living body.

3. The method according to claim 2, wherein the gene encoding the protein that interferes with the activity of the expression product is introduced into the living body via an expression vector consisting of a naked DNA.

4. The method according to claim 3, wherein the expression vector is introduced into the living body by electroporation.

5. The method according to claim 1, wherein the protein that interferes with the activity of the expression product is a protein selected from the group consisting of: a receptor of the expression product, an activity-neutralizing antibody of the expression product, and a protein containing an active site of the receptor or the antibody.

6. The method according to claim 2, wherein the gene encoding the protein that interferes with the activity of the expression product is expressed under the control of an inducible promoter.

7. A method for regulating the activity of an expression product of a gene introduced in gene therapy by the method according to claim 1.

8. The method according to claim 7, wherein the introduced gene encodes a protein which belongs to a group selected from blood proteins, cytokines, and hormones.

9. The method according to claim 8, wherein the protein belonging to a group selected from blood proteins, cytokines, and hormones is selected from the group consisting of:
erythropoietin, granulocyte-colony stimulating factor, granulocyte-macrophage colony stimulating factor, stem cell factor, thrombopoietin, tumor necrosis factor-α, transforming growth factor-β, CD4, CD45, bone morphogenetic protein, interferon-α, interferon-γ, interleukin-1, interleukin-2, interleukin-6, interleukin-11, interleukin-12, epidermal growth factor, acidic fibroblast growth factor, basic fibroblast growth factor, fibroblast growth factor-I, platelet derived growth factor, vascular endothelial growth factor, nerve growth factor, brain derived neurotrophic factor, insulin-like growth factor-I, insulin-like growth factor-II, human growth hormone, parathyroid hormone, angiostatin, pigment epithelial cell derived factor, and hepatocyte growth factor.

10. The method according to claim 9, wherein the protein is erythropoietin.

11. The method according to claim 10, wherein the protein that interferes with the activity of the expression product is a solubilized erythropoietin receptor.

12. The method according to claim 1, wherein the protein that interferes with the activity of the expression product is a fusion protein with another protein.

13. An expression vector for regulating the activity of an expression product of a gene introduced into a living body, which can express a protein that interferes with the activity of the expression product in the same living body.

14. The expression vector according to claim 13, wherein the protein that interferes with the activity of the expression product is a fusion protein with another protein.

15. A composition for regulating the activity of an expression product of a gene introduced into a living body, which comprises the expression vector of claim 13 as an active ingredient.

16. Use of an expression vector that can express a protein which interferes with the activity of an expression product of a gene introduced into a living body in the same living body, in the production of a composition for regulating the activity of the expression product of a gene introduced into a living body.

17. The use of the expression vector according to claim 16, wherein the protein that interferes with the activity of the expression product is a fusion protein with another protein.


SEQUENCE LISTING (3 sequences)

SEQ ID NO: 1 - DNA, 5565 bp, Homo sapiens
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattccaattccaccatggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgactacaaagacgatgacgataaataagtcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 2 - DNA, 5504 bp, Rattus norvegicus
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcctcgaatcggagatgggggtgcccgaacgtcccaccctgctgcttttactatccttgctactgattcctctgggcctcccagtcctctgcgctcccccacgcctcatttgcgacagtcgcgttctggagaggtacatcttggaggccaaggaggcagaaaatgtcacaatgggctgtgcagaaggtcccagactgagtgagaatattaccgtcccagataccaaagtcaacttctacgcttggaaaagaatgaaggtggaagaacaggctgtagaagtttggcaaggcctgtctctgctctcagaagccatcctgcaggcccaggctctgcaggccaattcctcccagccaccagagagtcttcagcttcatatagacaaagccatcagtgggctacgtagcctcacttcactgcttcgggtgctgggagctcagaaggaattgatgtcgcctccagacgccacccaagccgctccactccgaacactcacagcggatactttctgcaagctcttccgggtctactccaacttcctccgggggaaactgaagctgtacacgggggaggcctgcaggagaggggacaggtgacctgccactgccgtgtacccgccaactcgctcaccgtcactgtgtcacgccaaccctccaccactcccaaccctcatcaaacggggttgtttgttaccttcttaccggcctgtcctacgcggcctcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 3 - DNA, 6256 bp, Unknown
  Chimeric gene, pCAGGS-hSEPOR2 rat IgG1 Fc cDNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcatttaaataattccaccatggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggacgcggccgccgtgcccagaaactgtggaggtgattgcaagccttgtatatgtacaggctcagaagtatcatctgtcttcatcttccccccaaagcccaaagatgtgctcaccatcactctgactcctaaggtcacgtgtgttgtggtagacattagccaggacgatcccgaggtccatttcagctggtttgtagatgacgtggaagtccacacagctcagactcgaccaccagaggagcagttcaacagcactttccgctcagtcagtgaactccccatcctgcaccaggactggctcaatggcaggacgttcagatgcaaggtcaccagtgcagctttcccatcccccatcgagaaaaccatctccaaacccgaaggcagaacacaagttccgcatgtatacaccatgtcacctaccaaggaagagatgatccagaatgaagtcagtatcacctgcatggtaaaaggcttctatcccccagacatttatgtggagtggcagatgaacgggcagccacaggaaaactacaagaacactccacctacgatggacacagatgggagttacttcctctacagcaagctcaatgtgaagaaggaaaaatggcagcagggaaacacgttcacgtgttctgtgctgcatgaaggcctgcacaaccaccatactgagaagagtctctcccactctccgggtaaatgaccccagagtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg